(19) United States
(12) Patent Application Publication
(10) Pub. No.: US 2023/0187057 A1
(43) Pub. Date: Jun. 15, 2023

(54) SYSTEM FOR TRACKING THE STATE OF A HOSPITAL RESOURCE

(71) Applicant: CONSOMIX, PARIS (FR)

(72) Inventors: Meyer Patrick MELUL, PARIS (FR); Dann MELUL, TEL-AVIV (IL)

(21) Appl. No.: 17/923,795

(22) PCT Filed: May 17, 2021
(86) PCT No.: PCT/EP2021/063030

§ 371 Date: Nov. 7, 2022

(30) Foreign Application Priority Data
May 17, 2020 (FR) 2004910

(51) Int. Cl. G16H 40/40; G16H 40/67

(52) U.S. Cl. CPC G16H 40/40; G16H 40/67

(57) ABSTRACT

A system for tracking the state of a resource of an establishment includes a connected device including a physical actuator for the activation of at least one physical occupancy status of a resource with which the actuator is associated; a visual indicator of a chosen physical occupancy status of the resource; a system of attachment to the resource, and at least one calculator to extract a virtual occupancy status of a resource from the database, and record at least one change in the virtual occupancy status of a resource in the database from the reception of data including a physical occupancy status of the resource.

### FIELD

The field of the invention relates to the field of systems for tracking resources and their occupancy status in an identified pool of resources. More particularly, the field of the invention pertains to systems for tracking hospital resources.

### PRIOR ART

Currently, solutions exist making it possible to track hospital resources in order to monitor their occupancy and anticipate new patient is arrivals. Such solutions notably make it possible to identify free hospital beds in order to assign them to a patient.

However, current solutions are typically implemented by a centralized system in which a doctor or medical staff must first consult a computer remote from the room in which a presumed “free” bed is located to assign it to a future patient arrival. Thus, the doctor or medical staff member may make assignment errors, since there is no guarantee that the resource will be in line with expectations, i.e. free or in working order or equipped with a given material such as a ventilator, medical robot or any other type of medical resource. Further, hospital beds can be moved. Thus, in the hospital a resource that is thought to be in one place may not actually be there if it has been moved in the meantime.

Finally, current solutions are typically focused on managing a room of a patient. Thus, when different beds are in a same room, assignment errors can multiply. Similarly, when a bed is moved from a room, the resource management system must be re-configured to re-associate the resource with a new room and to re-establish the tracking thereof. These solutions require a tracking of the management system in order to ensure that a bed in the system indeed corresponds to that of a given room.

Yet, beds in hospitals are becoming more and more mobile and current systems thus often become obsolete. Finally, current systems most often implement a system for managing a resource, such as a bed, associated with a specific room, but cannot integrate different types of resources, such as a ventilator or a medical robot. However, it is sometimes necessary to coordinate the allocation of several resources to a patient or at least to plan or forecast allocations of a plurality of resources to a patient.

Another problem with existing resource management tracking solutions is the limitation of the accessibility of information on resource availability at the level of a single establishment. Thus, within the same hospital, resources are administered with patient information. It is then difficult for these solutions to separate “shareable” information from “non-shareable” information outside the hospital location because the information systems are built around the subject of the “patient” and “patient room”. In other words, current systems are not designed to share a part of their information with other establishments. As a result, staff of an establishment or a resource management organization cannot obtain data shared by one hospital and process it with data from other hospitals in order to anticipate needs quickly.

Finally, a limitation to this data exchange is that the information systems do not allow the sharing of up-to-date and reliable resource availability data in real time. A limitation is that updating the availability of a resource requires the use of a resource management interface far from the resource itself, while the latter may, in the meantime, be assigned to another need.

A general drawback resulting from the aforementioned problems is that existing solutions do not make it possible to optimize the time a resource is unoccupied in order to reduce it. Yet, this time can prove to be vital for proposing a welcome to patients in danger.

### SUMMARY OF THE INVENTION

The invention detailed below makes it possible to overcome the aforementioned drawbacks.

According to one aspect, the invention relates to a system for tracking the state of a resource comprising:
At least one connected device comprising:
   a physical actuator for the activation or selection of at least one physical occupancy status of a resource with which the actuator is associated, the physical occupancy status being selected from a set of physical occupancy statuses comprising at least three states among which a first availability status, a second occupancy status and a third status;
   a memory to store data from said connected device, of which at least one identifier of said connected device;
   a communication interface to exchange or transmit data stored in the memory of said connected device with at least one remote server;
   a visual indicator of a chosen physical occupancy status of said resource;
   a means of attachment to the resource so as to make said connected device integral with at least one resource;
at least one remote server accessible from at least one data network, said server comprising:
   a memory containing a database and/or data files referencing the different resources and/or different connected devices and virtual occupancy statuses;
at least one calculator to:
   extract at least one virtual occupancy status of a resource from said database and/or said data files and transmit a message within the data network comprising at least one virtual occupancy status of at least one resource;
   record at least one change in the virtual occupancy status of a resource in said database from the reception of data comprising a physical occupancy status of the resource, said data being transmitted by the connected device attached to said resource.

According to one aspect, the invention relates to a system for tracking the state of a resource comprising:
At least one connected device comprising:
   a physical actuator for the activation or selection of at least one physical occupancy status of a resource with which the actuator is associated, the physical occupancy status being selected from a set of physical occupancy statuses comprising at least three states among which a first availability status, a second occupancy status and a third status;
   a memory to store data from said connected device, of which at least one identifier of said connected device;
   a communication interface to exchange or transmit data stored in the memory of said connected device with at least one remote server;
   a visual indicator of a chosen physical occupancy status of said resource;
at least one remote server accessible from at least one data network, said server comprising:
   a memory containing a database and/or data files referencing the different resources and/or different connected devices and virtual occupancy statuses;
at least one calculator to:
   extract at least one virtual occupancy status of a resource from said database and/or said data files and transmit a message within the data network comprising at least one virtual occupancy status of at least one resource;
   record at least one change in the virtual occupancy status of a resource in said database from the reception of data comprising a physical occupancy status of the resource, said data being transmitted by the connected device attached to said resource.

One advantage of the invention is that it allows a better visualization of the occupancy state of a resource in an establishment, notably in contact with the resource, and a facilitated access to the means to change the availability of the resource in order to save time spent unoccupied by the resource. Another advantage is to enable this visualization and this access while leaving the possibility, for example, of reserving a resource remotely, notably thanks to the use of virtual occupancy status.

According to one aspect, the invention relates to a device comprising
a physical actuator to select at least one physical occupancy status of a resource with which the actuator is associated, the physical occupancy status being selected from a set of physical occupancy statuses comprising at least three states among which a first availability status (“available”), a second occupancy status (“occupied”) and a third status (“Neutralized”, “Out of Service”, “Awaiting Cleaning”);
a memory (Mo) to store data of said connected device of which at least one identifier of said connected device;
a means of attachment to the resource, to a wall or to a door in such a way as to make said connected device integral with at least one resource, wall or door respectively;
a communication interface to transmit data stored in the memory of said connected device with at least one remote server and to receive and store in the memory a message comprising a value of a virtual occupancy status transmitted by said remote server;
a visual indicator to indicate a state feedback representing the selected physical occupancy status; and
a calculator to generate an electrical set point to light up the visual indicator as a function of the virtual occupancy status received by the remote server.

According to another aspect, the invention relates to a bed, preferably a hospital bed comprising such a device according to the invention.

According to one embodiment, the resource is associated with a given establishment and at least one data network comprises at least one network equipment arranged within said establishment, said network equipment being a proxy or VPN concentrator to collect all of the messages transmitted by a plurality of connected devices. One advantage is to facilitate the network configuration of a pool of connected devices that would be deployed within an establishment

According to one embodiment, the data extracted from the database and/or data files comprising the virtual occupancy statuses are transmitted to:
at least one electronic terminal accessible from the data network and/or;
the connected device of which the physical occupancy status corresponds to the virtual occupancy status.

One advantage is to make accessible for medical staff the virtual status database from any terminal. One advantage of returning the modification of the virtual occupancy status to the connected device is to generate a state of the status that is visible on the device and optionally to modify the current physical occupancy status of the connected device if it is different.

According to one embodiment, the actuator is a rotary button comprising a visual indicator indicating the selected physical occupancy status. One advantage is to propose an easy-to-use actuator nor requiring a connection or digital interface with a system and which can continuously deliver a state feedback of the physical occupancy status.

According to one embodiment, the actuator is a button making it possible to select a physical occupancy state, said button comprising a visual indicator making it possible to indicate a state feedback of a modification of a virtual occupancy status. One advantage is to enable a state feedback that can be controlled according to a set point not dependent on the position of the button for example from the network, said set point representing a state of a virtual occupancy status. According to one embodiment, the visual indicator comprises a diode or consists of a diode.

According to one embodiment, the connected device comprises an interface allowing access control of an individual, the access control making it possible to initiate a modification of the physical occupancy status according to a predefined value among all the values of the physical occupancy status. One advantage is to limit access to certain statuses, such as the “neutralized” status, or certain functions, such as the parameter setting mode, to personnel having sufficient access rights.

According to one embodiment, the connected device comprises a return screen on which at least one datum is displayed, said datum being transmitted by a remote server. One advantage is to make it possible to display data from the server, such as a patient identifier.

According to one embodiment, the number of virtual occupancy statuses is strictly greater than the number of physical occupancy statuses. One advantage is to enable status management such as the reservation, the allocation, the transfer of a resource without requiring an immediate physical occupancy status change at the level of the connected device linked to the resource.

According to one embodiment, at least one value of a physical occupancy status corresponds to a first value of a virtual occupancy status or a second value of a virtual occupancy status. One advantage is to make it possible to have available a status at the resource level such as “available” while leaving the possibility from the point of view of the application to manage different states of this physical occupancy status, notably thanks to the definition of virtual occupancy statuses such as “unallocated” or “allocated”.

According to one embodiment, the “available” value of the physical occupancy status corresponds to one of the two values {“allocated”; “unallocated”} of the virtual occupancy status.

According to one embodiment, the modification in a remote database or a remote file of the value of a virtual occupancy status leads to an automatic generation of a request transmitted through at least one data network to the connected device, said connected device comprising a calculator to generate an electrical set point to light up a visual indicator such as a diode of said actuator. According to one embodiment, the generation of the set point also leads to a modification of the physical occupancy status. One advantage is to make it possible to notify a medical staff member in the vicinity of the resource that the virtual status has changed. Thus, the medical staff member can initiate an action on account of the lighting up of a diode. For example, he can apply a management rule linked to this new virtual status to this resource, finalize the preparation of a resource or change the position of the actuator.

According to one embodiment, in the case where the diode represents the physical occupancy status, the change in the state feedback of the diode causes the modification of the physical occupancy status. This case occurs, for example, when the position of the button is the identical whatever the physical occupancy status value. This is the case, for example, for a push button with a position return to the initial state of the button. It is understood in this case that the value of the physical occupancy status is indicated by the state of the diode.

According to one embodiment, the means of attachment of the connected device to a resource comprises two strips cooperating together, a first strip comprising loops and the second strip comprising hooks. One advantage is to have available an attachment that is able to adapt to different types and sizes of resource supports.

According to one embodiment, the connected device comprises a geolocation module configured to receive a location message, said location position being recorded in a memory of said connected device and transmitted to a remote entity. One advantage is to make it possible to track roaming resources, such as ambulance resources. Another advantage is to make it possible to track a position of a resource being transferred from one establishment to another.

According to one embodiment, the system comprises:
a first server configured to collect the data transmitted by the connected devices of a first data network,
a second server accessible from a second data network connected to the first data network, configured to collect part of the data collected by the first server of the connected devices of the first data network.

One advantage is to make it possible to feed back availability information to a platform that is accessible to different establishments without having to feed back unnecessary patient data or status data, such as “awaiting cleaning”, to access the availability of a resource pool.

According to another aspect, the invention relates to a method for tracking the state of a resource comprising:
Automatic transmission of a data message from a device connected to a data network following the detection of a change in position of an actuator, said message comprising a physical occupancy status of a resource associated with said connected device;
Recording at least one change in the virtual occupancy status of a resource in said database from the reception of data comprising a physical occupancy status of the resource;
Extracting at least one virtual occupancy status of a resource from said database and transmission of data to an electronic terminal accessible from the data network, said data comprising at least one virtual occupancy status of at least one resource;
Modification of at least one change in the virtual occupancy status of a resource in said database from the reception of a command transmitted from an electronic terminal connected to the data network, said command comprising at least one value of a virtual occupancy status comprised in a set of values, said set of values corresponding to a value of a physical occupancy status of the resource.

One advantage is to enable an update of the virtual occupancy statuses in a data file or database while keeping an actuator to modify a physical occupancy status of a resource that corresponds to different values of the virtual occupancy status.

### BRIEF DESCRIPTION OF THE FIGURES

Other characteristics and advantages of the invention will be become clearer on reading the following detailed description, with reference to the appended figures, that illustrate:

FIG. 1: a hospital bed equipped with a connected device of the invention according to one embodiment of the invention;

FIG. 2: an example of the arrangement of one embodiment of a connected device of the invention on a ventilator;

FIG. 3: a first example of a rotary actuator having an adaptive attachment of a connected device of the invention;

FIG. 4: the first example of a rotary actuator in another attachment position than the position of the connected device of FIG. 3;

FIG. 5: a second example of an actuator of a connected device of the invention;

FIG. 6: an alternative of the second example of an actuator of a connected device of the invention;

FIG. 7: an example of a single push button actuator of a connected device of the invention;

FIG. 8: one embodiment of a system of the invention.

In the remainder of the description, the term “physical occupancy status” STi is understood to mean a usage status of a resource such as a bed or ventilator that can be directly selected from the connected device attached to a resource and of which the result of the selection is directly visible on said connected device. The selection of a physical occupancy status STi has for effect the generation and transmission of a data message from the connected device to a remote server in order to modify the value in a database or a file centralizing the data relating to the status of a resource pool.

In the remainder of the description, the term “virtual occupancy status” is understood to mean a usage status of a resource such as a bed or ventilator that can be accessed or viewed from an electronic terminal, such as a tablet, phone, computer, server, said electronic terminal being remote from the connected device or said resource. Some virtual occupancy statuses can be selected within a user interface of an electronic terminal. In this case, the result of the selection has as a consequence the transmission of a data message to a remote server to update the virtual occupancy status of the resource in a database or a file centralizing the data relating to the status of a resource pool.

According to one embodiment, the selection of a virtual occupancy is status STi′ has the effect of automatically sending a data message from an electronic terminal or from a server to the connected device in order to modify the visibility of the status directly on said connected device when the latter is equipped with a state feedback. The state feedback has the effect of modifying the physical occupancy status STi or encouraging or inviting it to be modified by a person present by a predefined state feedback. According to another embodiment, the selection of a virtual occupancy status STi′ does not have as effect a change in the physical occupancy status STi.

Consequently, the physical occupancy status STi can be indicated by a visual indication directly legible on the connected device and a digital datum transmitted to a remote server. The virtual occupancy status STi′ may be indicated by a digital datum transmitted to a remote server and optionally by a digital datum transmitted to the connected device to modify the visual indication relating to the status directly readable on the connected device.

Attachment to a Resource

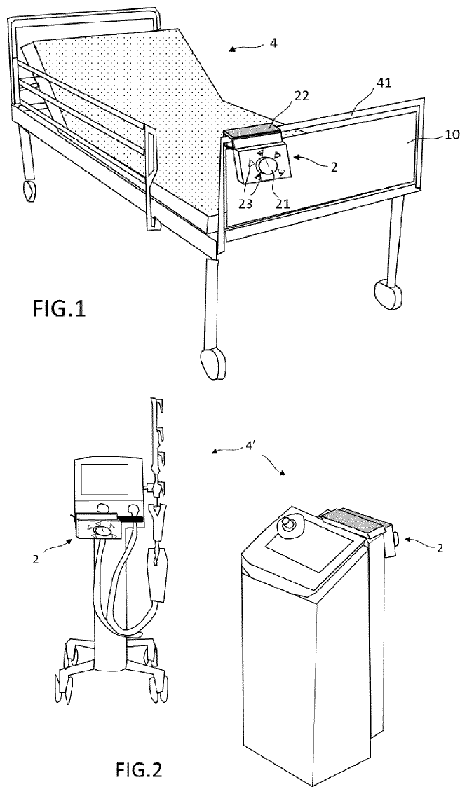

FIG. 1 shows a resource 4, such as a bed, to which a connected device 2 of the invention is attached. The resource 4 is, for example, a hospital bed and it is thus located in a hospital-type establishment. However, the establishment is a place where the resource is used. Nevertheless, the bed can be transported and moved from one establishment to another by means of a lorry, ambulance, train or plane. The invention is thus not limited to the scope of the establishment since the connected device 2 of the invention can transmit information to the system 1 from the moment that a connection to a data network such as the Internet is possible.

A hospital bed is understood mean, in a non-restrictive manner, a bed comprising mechanical elements intended for medical use. The mechanical elements may be, for example, in a non-limiting manner, a side rail, casters, a brake, an adjustable mattress platform, a footboard comprising a first frame or a headboard comprising a second frame. They may also be electronic elements such as a remote control to actuate a motor, a screen, mains sockets or computer connections.

In this scenario of FIG. 1, the connected device 2 is attached to a footboard frame 4. In other configurations, the connected device 2 is attached to a side frame of the bed or to the headboard frame. According to an example, the device is arranged in a compartment of the bed or in a bag attached to the bed 4 in such a way as to be integral with said bed.

According to one embodiment, the device may be attached to a resource irreversibly such as with rivets or screws.

According to another embodiment, the device may be integrated into said resource. For example, a hospital bed may comprise such a device according to the invention incorporated in the bed. The device may be integrated in the bed frame, in the headboard, in the remote control of a medical bed. Notably, the actuator may be arranged on the surface of the frame of a bed in such a way that it is protected against impacts of the bed against the walls when it is moved.

According to another embodiment, the connected device may be remote. For example, the device may be arranged on the surface of a room wall or door. This embodiment advantageously allows a resource to be moved without moving the device. Depending on the case, a resource move will not require disconnecting the electrical cable from the device or the wire link from the device. According to another embodiment, the resource is a room such as a bedroom or operating room, and the device is attached to the surface of a wall or door of the room.

According to one embodiment, the connected device comprises means for attachment to a wall and/or a door of a room.

According to another embodiment, the resource is a health vehicle such as an ambulance. According to one embodiment, the connected device comprises means of attachment to a wall and/or a door of a health vehicle.

According to another embodiment, the visual indicator of the connected device may be remote. For example, the visual indicator may be arranged on the surface of a wall or door. This embodiment advantageously makes it possible to alert medical staff without having to go into the room to know if a resource is available or not, requiring them to go back into the room to modify the physical status to limit errors when the physical actuator is attached to the resource or integrated into said resource.

In one embodiment, the device may comprise two or a plurality of visual indicators, such as light indicators, all arranged in different locations. This embodiment advantageously makes it possible to view the physical status of the resource from different locations.

According to an example, the connected device 2 is provided with a means of attachment 22 that adapts to different types of resources and thus beds 4. The connected device 2 may comprise a universal type attachment, i.e. one comprising an adaptive means making it possible to be made integral with different sizes of mounting supports. Such an attachment may be a hook-and-loop type fastener so as to wrap a support or an attachment clamp forming an adjustable “U” and comprising a means of exerting a return force to grip a support. One advantage is to enable an attachment of the connected device 2 to different types of resources 4, 4′ and to a wide variety of types of the same resource.

FIG. 2 shows a ventilator 4′ that may be another type of resource to which the system 1 of the invention refers. In the case of a ventilator 4′, the connected device of the invention may be attached to the chamber comprising the oxygen tank or to the control and interface unit allowing oxygen to be delivered to a patient. FIG. 2 shows the two units and two methods of attaching the connected device 2. In the first case, the connected device 2 is attached to the back of the chamber comprising an oxygen tank and, in the second case, the connected device 2 is attached to an attachment bar on the control unit of a ventilator 4′. Other arrangements and methods of attachment are possible according to other embodiments of the invention. A room may also be another type of resource that the system 1 refers to, as well as an operating room or any other type of room that could constitute a hospital resource.

Actuator

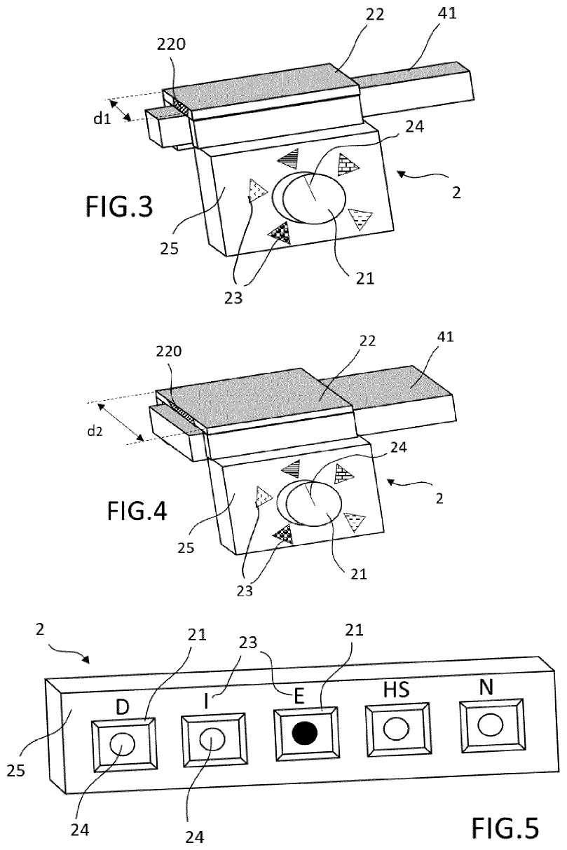

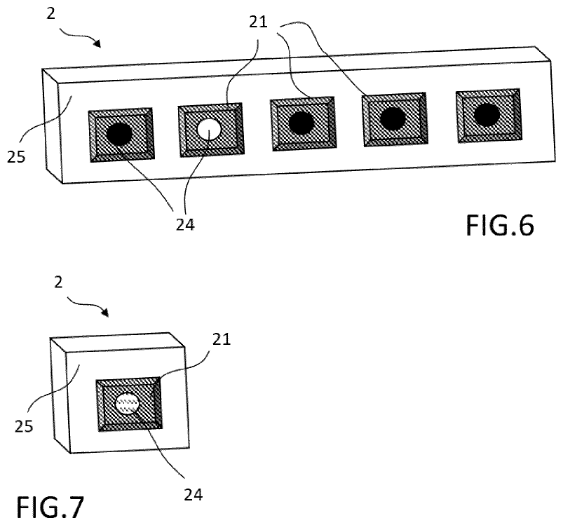

FIG. 3 shows an embodiment of a connected device 2 of the invention comprising a rotary button 21 type actuator also known as a “rotary control”. Such a rotary control forms a rotating electrical contactor connecting circuits to different electrical or electronic tracks. Each electrical track is associated with a value of a physical occupancy status of the resource. The rotary button 21 comprises a position marking 24 making it possible to position and reference the positioning of the rotary button 21 on a marking 23 of a frame of the connected device 2. In the case of FIG. 3, 5 markings 23 make it possible to select a status value out of 5 possible. One interest is that the selected status is immediately visible by looking at the front face of the connected device 2. There is an interest for medical staff to immediately is identify if a resource is available, out of service, awaiting cleaning, or neutralized. Generally, medical staff can visually assess whether a bed is occupied or not, but it is possible that the bed is occupied and the patient is temporarily absent. Finally, when the resource is a ventilator, it is more difficult to know its situation when the latter just been used by a team or has just been prepared to be transported to a patient. There is thus an interest to position the occupied status and to visually asses the status of the button to confirm the information. According to another example, the actuator is a slide switch.

Generally speaking, the actuator may be a mechanical actuator in so far as the action undertaken causes the modification of a mechanical state of the actuator, as is the case of a rotary button or a series of switches.

According to one embodiment, the physical actuator is a touch-type actuator that combines display functionality and pointing functionality. An interest is to return a state of the actuator while allowing actuation of a command. According to this example, the pointing functionality of the touch actuator is ensured by a technology of the capacitive or inductive or optical or infrared type or any other technology making it possible to generate a command from a touch of an interface forming the actuator.

The connected device 2 of FIG. 3 also comprises an adaptive attachment 22 so that it is adjustable to different thicknesses d1, d2 of a support 41. In the case of FIG. 3, the attachment 22 is adjusted to grip a support 41 of a resource 4, 4′ in its width. The gripping of the support 41 may, for example, be achieved thanks to a device 220 that exerts a recoil force to bring two edges of the attachment towards each other. In the case of FIG. 4, the same connected device 2 as that in FIG. 3 is adjusted to be attached to a support 41 of a width d2 wider than in the exemplary case of FIG. 3.

The rack 25 may be equipped with visual indicators such as markings, however in other embodiments, the visual indicators 23 may be diodes, the lighting of which indicates the position of the rotary control 21.

According to another embodiment, the rack 25 of the connected device 2 comprises no marking and it is only the rotary control 21 that comprises a diode-type indicator. It may comprise a single diode, the color of the lighting then indicates the status value. According to an alternative, it may comprise several diodes, each illumination of which indicates an exclusive binary status of the other statuses.

In one exemplary case, the diode is coupled with a shutter forming an opening to let light pass through according to an inscription or shape. For example, the shutters may form openings in the form of letters: “HS”; “N”; “D”; “O”; “A” may indicate for example the statuses: “Out of Service”, “Neutralized”, “Available”, “Occupied”, “Awaiting Cleaning”.

FIG. 5 shows another embodiment of a connected device 2 of the invention comprising an actuator comprising a plurality of push buttons 21. In this case, each push button 21 makes it possible to select a given physical occupancy status STi. Pressing a push button 21 leads to the selection of a new physical occupancy status STi corresponding to the position of the push button 21 and automatically leads to the deselection of another physical occupancy status STi or a virtual occupancy status STi′ having previously been selected corresponding to the position of another push button 21. Thus, in this embodiment, the push button 21 corresponds by association with a predefined physical occupancy status STi. The color of the push button 21 or an inscription 23 on the rack 25 of the connected device 2 or directly on the front face of the push button 21 indicates this association. As in the case of FIG. 4, these may be inscriptions 23 forming letters or a symbol for example.

According to one embodiment, each button 21 comprises a diode 24 making it possible to deliver a feedback state of the selection of a given physical occupancy status STi. In this embodiment, one advantage is that the modification of a virtual occupancy status STi′ may be taken into account by the connected device 2. The push button being for example a button in a trigger state with a single return position, in other words it cannot remain pressed and returns to its initial position after pressing. This option makes it possible to disregard physical button positions, but only changes of state of the statuses. One advantage is that the taking into account of a virtual occupancy status STi′ does not imply a modification of the physical position of the button.

FIG. 7 shows an even simpler actuator with a single push button 21, the number of presses of which circularly determines a state of a status. When 5 statuses are used, it is possible to change the status by pressing the button a maximum of 5 times to scroll through all the possibilities from any status. The diode 24 may then be used to visually represent one of the 5 statuses. In this configuration, when a virtual occupancy status STi′ is received, the diode 24 indicates the value of said new virtual occupancy status STi′ which becomes the new physical occupancy status STi. A new sequence of changes of physical occupancy status STi from the actuator 21 is then possible starting from the physical occupancy status STi that has just been modified on the connected device 2.

According to an alternative to FIG. 7, a single push button 21 or more generally a single actuator may be associated with 5 diodes which may for example be aligned. Successive pressings of the push button 21 makes it possible to change the status in turn, the diodes make it possible to give to a user a state feedback of the selected status.

Alternatively, two push buttons 21 may be present so that pressing one of the two leads to a change of the statuses and diodes to the right or left, for example, if they are aligned horizontally and pressing the other push button 21 changes the statuses and diodes respectively to the left or right. Another example with two buttons making it possible to move the light of the diodes vertically is also possible if the diode alignment is vertical.

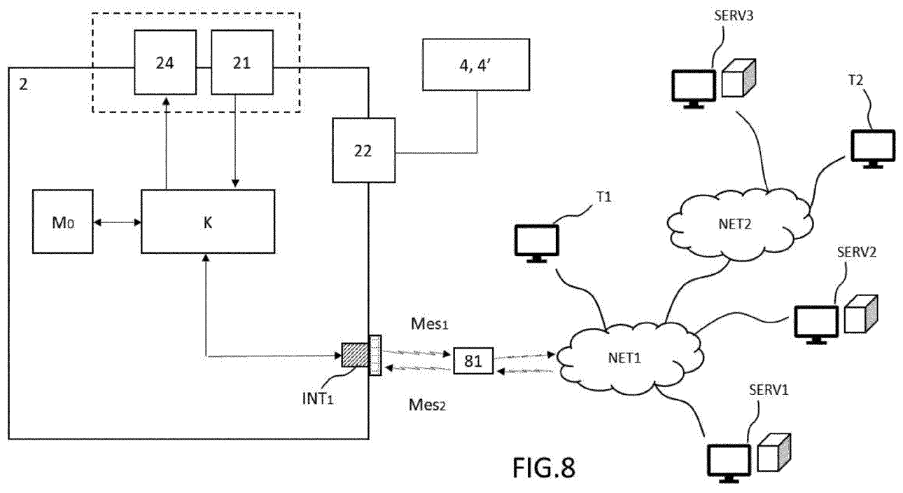

FIG. 8 shows an embodiment of a connected device 2 of the invention and a possible exemplary architecture of the system of the invention.

Connected Device

The connected device 2 comprises at least one memory to store at least one datum corresponding to an identifier ID₂. According to one embodiment, the connected device 2 comprises a memory to store network data such as server addresses, passwords, protocol data allowing it to address data messages according to different data protocols. According to an example, a control software is installed in a memory of the connected device 2. For example, a pre-installation makes it possible to instantiate a boot version of the on-board software of the connected device 2. According to an example, a first pre-installation may correspond to the installation of a first software version in which a network configuration takes into account the presence of a proxy or a VPN concentrator, a second pre-installation may correspond to the installation of a second software version in which a network configuration does not take into account the presence of a proxy or a VPN concentrator.

This pre-installation enables a facilitated configuration of the connected device the first time it is used.

According to an example, configuration data are recorded in a memory of the connected device, for example a default connection address, a is default password or data related to the type of software version embedded in the connected device 2 or data related to the physical version of the rotary control and state feedback indicators.

According to an example, the default values of the statuses are recorded in the memory of the connected device 2. The connected device 2 may comprise a single memory Mo making it possible to store all the data necessary for its operation or it may comprise a plurality of memories making it possible to record different data in different components of the connected device 2.

The connected device 2 comprises a calculator K notably making it possible to:
Receive a digital set point of change of state of a physical occupancy status STi from an actuator 21;
Automatically generate a data message Mes1 comprising a value of a physical occupancy status STi to a remote entity of a data network such as a server SERV₁ or SERV₃;
Generate digital set points to a diode 24 or a display of the connected device 2;
Record data in a memory Mo of said connected device which are either received in a data message Mes2 through a data interface INT₁ from an equipment of a data network, or received from an actuator 21.

According to one embodiment, the connected device 2 comprises a battery unit. According to one embodiment, the battery unit is rechargeable. According to one example, the battery unit is removable by a quick-release attachment of clip type so that it can be replaced. According to one example, the battery unit contains at least one battery. When the battery unit is replaced, the last physical occupancy status STi in the memory is displayed again by means of the state feedback of said connected device 2.

According to one example, the connected device 2 comprises a power supply interface making it possible to electrically connect said connected device 2 to an energy source of a room. According to another example, the connected device 2 comprises a power supply interface to electrically connect said connected device 2 to an electrical circuit of a is resource 4 or 4′. According to an example, the power supply connector is of USB connector type.

According to one embodiment, the device further comprises a state confirmation user interface. The state confirmation user interface may comprise a push button, such as a push button with a return of position to the initial state of the button. One advantage is to be able to confirm, by hospital staff passing in front of the resource, that the physical status corresponds to the actual status of the resource. The user confirmation interface generates a confirmation datum that may be transmitted within the data network via a data message. One advantage is to be able to reduce errors, to avoid a patient being forgotten, and to trace the visits of hospital staff. With this in mind, the user confirmation interface may also comprise a means of authentication, for example a magnetic badge reader making it possible to identify the cardholder.

Remote Control

According to one embodiment, the connected device 2 may be remotely controlled by a means of a remote control. The remote control makes it possible to change the status of the device or communication parameters such as the WiFi password. In this embodiment, the remote control thus comprises an actuator making it possible to change the physical occupancy status STi. According to an example, the remote control is a detachable part of the connected device 2 which also forms its front face.

According to one embodiment, a plurality of remote controls makes it possible to send commands or set points to a connected device 2. This option makes it possible to deliver remote controls to medical personnel who can act on different connected devices 2. This action is then made possible after identification of the connected device of a user having a remote control. The identification may notably be carried out, as described below, by a magnetic badge, the establishment of a near field connection or any other means of delivering identification and possibly authentication data to the connected device.

Communication Interface of the Connected Device

According to one embodiment, the system of the invention comprises the connected device 2 and at least one network equipment 81. The connected device 2, by means of at least one communication interface INT₁, communicates locally with the network equipment 81. The latter may be a modem, a WiFi terminal, a router, a VPN concentrator, a Proxy or any other network equipment making it possible to ensure communication. The link between the connected device 2 and the network equipment 81 is for example of Ethernet, WiFi, LoRa type, etc. and more generally a wired or wireless link or a combination of wired or wireless links.

For example, the network equipment 81 may comprise one proxy or two redundant proxies so as to secure access when one of the two proxies fails.

According to one embodiment, a proxy ensures an encoding and an encryption of the data transmitted over at least one data network such as NET₁ or NET₂.

One advantage of the use of a proxy is to facilitate the network administration by for example avoiding managing communication parameters for each of the connected devices 2, such as an encryption key, which although defined at the proxy level makes it possible to encrypt the data transmitted by the devices 2 on a public network such as the Internet.

According to one embodiment, the network equipment 81 is a Smartphone, a tablet or a 3G, 4G key connected to a computer or a 3G or 4G connection box. In this case, the link between the connected device 2 and the network equipment 81 is USB, Ethernet, Bluetooth, WiFi, LoRa, or any other near field protocol.

According to a first installation, a network access by means of a Bluetooth connection makes it possible to configure network parameters such as a WiFi password to the connected device. Then the connection is established consecutively by the WiFi network.

According to another example, a network access by means of a previously configured WiFi connection with default parameters makes it possible to configure network parameters such as a WiFi password to the connected device. The connection is established consecutively by the WiFi network.

According to an exemplary embodiment, the connected equipment 2 comprises an access to a parameter setting mode from a control interface of a screen or from a specific button. This parameter setting mode makes it possible for example to define a WiFi password and choose a specific WiFi network, for example.

According to one embodiment, the network equipment 81 is associated with an establishment or an area of an establishment such as a floor or a room.

According to one embodiment, the network equipment 81 makes it possible to exchange data within a data network NET₁ and/or NET₂ with on the one hand at least one connected device 2 and on the other hand at least one network equipment such as a data server such as SERV₁, SERV₂, SERV₃ or any other network equipment.

According to an exemplary embodiment, some of the messages transmitted by each connected device 2 of the system 1 are “heartbeat” type messages that are transmitted at regular intervals. Thus, the system 1 is configured to generate alerts when a connected device 2 has not transmitted any messages for a certain time, which would seem to indicate that the connected device 2 is broken down, disconnected, or that its battery unit needs to be replaced.

Indeed, in some embodiments and under certain circumstances, messages transmitted by a connected device 2 may be lost or received with a delay to the SERV₁ server. “Heartbeat” type messages, transmitted at regular intervals by a connected device 2, make it possible, among other things, to compensate for the loss of a message transmitted by a connected device 2 during a change of physical status STi.

Association between a connected device and a resource According to one example, when a connected device 2 is associated for the first time with a resource 4, 4′, a step of associating an identifier ID₂ of a connected device 2 with an identifier ID₄ of a resource 4, 4′ is carried out. This association makes it possible to manage within the system 1 of the invention a single entity {connected device; resource}. When reassigning a connected device 2 to a new resource 4, 4′, a new entity formed by the association of two new identifiers is realized.

Initialization

According to one embodiment, the association between an identifier ID₂ of a connected device 2 with an identifier ID₄ of a resource 4, 4′ is facilitated by an initialization mechanism. According to one embodiment, when a connected device 2 is switched on, a calculator automatically generates, to a remote entity SERV₁ or SERV₃, an initialization data message comprising at least the identifier ID₂ of the connected device 2 and an indicator, such as a “flag”, “Init” indicating that it is entering the initialization mode. For a predefined time, e.g. a few minutes, the connected device 2 transmits a plurality of times the “Init” type messages successively. Under these conditions, a user may select said connected device 2 from a list of connected devices in “init” mode not yet assigned to a resource 4, 4′. This selection may be made by means of a user interface of an electronic terminal of terminal type T₁. One interest is that this association is carried out within an environment in which the resources and the connected devices may be identified digitally. The selection of a connected device 2 makes it possible to assign it in the system of the invention to the resource to which it is physically connected.

According to another example, the transmission of a series of “Init” messages is triggered by pressing a push button present on the connected device 2.

According to one embodiment, the initialization message comprises the physical occupancy status STi indicated by the position of the actuator 21 at the time of initialization. One advantage is to transmit, at the same time as the initialization phase, the physical occupancy status STi of the connected device 2 so that it is immediately taken into account in the database DB₁.

QR Code or Identification Label

According to an example, the connected device 2 or the resource 4.4′ are provided with a graphic representation encoding information such as a bar code, QR code, Datamatrix, a flash code or any other graphic symbol making it possible to be decoded by a shape recognition software. The graphic code may comprise an access link to a data server such as the server SERV₁ allowing direct access to the parameters of said connected device 2 or said resource 4.4′ and facilitating maintenance operations such as registration of a new connected device 2 or a new resource 4.4′.

According to an exemplary embodiment, the acquisition of the graphic code is performed by an optic, such as a camera, smartphone, tablet or connected equipment. According to another example, the connected device 2 or the resource 4, 4′ has a label indicating their identifier ID₂, respectively ID₄, enabling a search to be carried out in a data server such as SERV₁ and to access the parameters of said connected device 2 or said resource 4,4′.

According to one embodiment, the association between an identifier ID₂ of a connected device 2 with an identifier ID₄ of a resource 4, 4′ is facilitated by the acquisition of a graphic code present on the connected device 2 allowing access to the parameters of said connected device 2 by means of an access link to a data server such as SERV₁. Then, it suffices to complete the association by entering the identifier ID₄ of the resource 4, 4′ to which the connected device 2 is attached, or by selecting it from a list of resources already defined.

According to one embodiment, the association between an identifier ID₂ of a connected device 2 with an identifier ID₄ of a resource 4, 4′ is performed in at least two steps including first an acquisition of a graphic code present on the connected device 2 allowing direct access to the parameters of said connected device 2 by means of an access link to a data server such as SERV₁, then acquisition of a graphic code present on the resource 4, 4′ to which the connected device 2 is attached, making it possible to associate the identifier ID₄ of this resource 4, 4 with the connected device 2.

Association at the Device Level According to one embodiment, the association between an identifier ID₂ of a connected device 2 with an identifier ID₄ of a resource 4, 4′ may be performed at the level of the connected device 2. According to an example, the connected device 2 is provided with a keyboard making it possible to record at least the identifier ID₄ of a resource 4, 4′ to which the connected device 2 is attached. The connected device 2 may also be provided with a screen allowing the user to view the buttons he selects. According to one embodiment, the connected device 2 then memorizes its own identifier ID₂ and the identifier ID₄ of the resource 4, 4′ with which it is associated. When transmitting at least one type of message to a server SERV₁, said server is informed of the association and can store said association in a memory or in a database in order to form a single entity composed of the two identifiers {ID₂, ID₄}.

Processing Messages Transmitted by the Connected Devices

According to one embodiment, the system 1 of the invention comprises a plurality of connected devices 2, each connected device 2 being attached to a resource 4, 4′ of at least one establishment such as a hospital. According to one embodiment, a server SERV₁ is configured to receive data messages transmitted by the connected equipment. According to an example, is the server SERV₁ is physically in the establishment, however there is nothing to prevent this server from being physically at another site. According to another embodiment, the different functions of the server SERV₁ may be implemented according to an architecture distributed within a plurality of servers. According to another embodiment, the server SERV₁ is located in the Cloud, for example within the Internet network. According to one embodiment, a server SERV₁ is configured to receive data messages transmitted by the connected equipment 2 of a same hospital. According to another embodiment, a server SERV₁ is configured to receive data messages transmitted by the connected equipment 2 of several hospitals.

According to an example, the server SERV₁ makes it possible to centralize data from different hospitals. According to one embodiment, the servers SERV₁ are configured to collect data from a hospital and to synchronize or transmit part of the data to a central server SERV₃ in a second step. One advantage is to make it possible to manage, at a server SERV₁ level, confidential data at an establishment level such as patient names or logistic data concerning the status of resources, such as the number of beds being cleaned within a hospital.

The server SERV₁ comprises a first function that aims to receive data transmitted by the connected devices 2 of the invention and to process them. To this end, the server SERV₁ comprises at least a calculator and a memory.

When the server SERV₁ decodes the data of a message Mes1 transmitted by a connected device 2 of the invention, a new value of the physical occupancy status STi is liable to be associated with this message. In this case, the server SERV₁ identifies the new physical occupancy status STi and records the new value of the corresponding virtual occupancy status STi′ in a memory.

According to one embodiment, the virtual occupancy statuses STi′ are recorded in a memory, for example in the form of files or databases DB₁. According to one embodiment, the memory is a memory of the server SERV₁. According to another example, the memory centralizing the values of the virtual occupancy statuses STi′ of the connected devices 2 is a remote memory.

The server SERV₁ may be configured so as to update the values of the virtual occupancy statuses STi′ with the value received from the physical occupancy status STi received from a connected device 2. One advantage is to maintain a single reference of the virtual occupancy statuses STi′. Another advantage is that when a bed is, for example, out of service, its “Out of Service” status may be updated by a simple action on the connected device 2 attached to the resource 4, 4′. Thus, the physical occupancy status STi of a resource 4, 4′ and thus its availability may be immediately shared by medical staff located directly near the resource. In the event that a resource is moved from a room, floor of the establishment or even from the establishment, the status of the resource may be updated at any time and anywhere. Thus, the system 1 of the invention makes it possible to conserve a continuity of connection as long as the connected device 2 remains connected to the network. Thus, the pool of connected devices 2 and therefore resources 4, 4′ is easily maintained and special operations do not affect the system for tracking a resource 4, 4′. According to one embodiment, when changing the location of a resource 4, 4′, for example room or establishment, a user can modify in the system of the invention, for example with the server SERV₁ or SERV₃, a new association between the resource and a room number or an establishment number.

For example, when a resource 4, 4′ equipped with a connected device 2 is introduced into a new establishment, a network equipment such as 81 is capable of routing data messages transmitted by the connected device 2 to the servers SERV₁, SERV₂, or SERV₃.

Transfer of a Resource

According to one embodiment, the transfer of a resource from one location to another, such as from a first hospital to a second hospital, is performed according to a sequence of steps comprising modifications of virtual occupancy statuses.

When scheduling a transfer of a resource 4, 4′, a medical staff member modifies the physical occupancy status ST₁ so that its value is “neutralized”. At this stage, the “neutralized” physical occupancy status is returned to the system 1 thanks to the connected device 2. Then, from an electronic terminal, such as T₁, connected to a data network, the same or another medical staff member modifies the virtual occupancy status STi′ so as to specify it. For example, the virtual occupancy statuses corresponding to the is “neutralized” physical occupancy status are:
“Neutralized—Intramural”
“Neutralized—In Transit”
“Neutralized—Transfer Accepted”
“Neutralized—From”

The status may be linked to information about the place of origin or destination which may be defined by the user and for example selected from a predefined list of establishments.

According to an exemplary embodiment, the following steps may be performed by means of the system of the invention:
A first staff member at a first establishment, for example Bichat Hospital, modifies the virtual occupancy status of a resource to the value “In transit to”.
The first staff member selects a first establishment, for example the “Cochin” Hospital, in order to define the destination of the resource.
A message is automatically sent via a calculator and a communication interface of the system 1 to a second staff member, such as a department head of the establishment n° 2, in the example “Cochin Hospital”
The transmitted message is received by a second staff member in the establishment n° 2 within a system interface. The second staff member sees, in the list of their resources, a resource with a “Neutralized—From” status associated with information from the source location: “Bichat”.
If the second staff member accepts or approves this request, a new message is sent to the first staff member at the first establishment, in the example Bichat Hospital.
Upon receipt of this validation, the resource automatically switches to a virtual occupancy status: “Neutralized—Transfer Accepted”, associated with the destination: “Cochin” and the status at Cochin changes to “Neutralized—Transfer Accepted”, associated with the origin: “Bichat”;
A medical staff member from the first establishment then physically sends the resource to the Cochin Hospital establishment.
When the resource is received in Cochin, a medical staff member performs a parameterization of said resource, for example to indicate its new location (floor, room, wing, etc.)
The medical staff member of the second establishment then modifies the virtual occupancy status to “Neutralized—Intramural”.

The resource is then ready for use in the second establishment.

Geolocation

When the connected device 2 comprises a geolocation component, the position of the connected device 2 may be taken into account to verify that a connected device 2 is indeed assigned to a new establishment.

According to one embodiment, the connected device 2 equipped with a geolocation component is associated with an ambulance resource, thus enabling the status of the ambulance resource and its geographical position to be known.

Multi-Establishment Architecture

According to an exemplary embodiment, the virtual occupancy statuses STi′ of the equipment of an establishment may be collected within a system controlling the tracking of a pool of connected devices of several establishments. For this purpose, the data corresponding to the virtual occupancy statuses STi′ of an establishment are transmitted to a remote server SERV₃. According to one embodiment, the centralization of all the data of the virtual occupancy statuses STi′ of all the connected equipment 2 from different establishments is performed directly from a central server SERV3.

The invention may therefore comprise a first centralized architecture allowing the management and tracking of the virtual occupancy statuses STi′ of the entire pool of connected devices 2.

According to another embodiment, the invention comprises a second “cascade” architecture comprising the collection and tracking of virtual occupancy statuses STi′ of an establishment which is, in a second step, shared or synchronized with a central server SERV₃ collecting data from different servers SERV₁ of different establishments.

One advantage is to make it possible to share the data on virtual is occupancy statuses STi′ between the different establishments. Thus, it is possible from one establishment to view the state of the virtual occupancy statuses STi′ of the resources 4, 4′ from another establishment in order to plan a redirection of patient(s) or a transfer of resource(s) from one establishment to another. In this case, from an electronic terminal such as a computer or tablet or smartphone, a user of one establishment may request a reservation or transfer of resources remotely from another establishment. When the request is received within the establishment in question, a resource allocation may be performed directly within an electronic terminal managed from the establishment or from the connected device 2 in question. According to an example, a medical staff member can then modify the position of the rotary control of the connected device 2 corresponding to the resource 4, 4′ in question to the “neutralized” position with a view to a transfer of said resource to another establishment. Another advantage is to make it possible to share global statistics such as the number of beds available in one hospital, without having to disclose all the virtual occupancy statuses STI′ of a given hospital to medical staff from another hospital. For example, this could be the number of resources 4, 4′ being cleaned. Thus, the architecture of a system 1, comprising a central server SERV₃ collecting data filtered by intermediate servers SERV₁, makes it possible to organize an optimized sharing of resource availability data. It can also be used to filter patient data or personal data that does not need to be shared and which is sometimes associated with data characterizing occupied resources.

Allocation of a Resource

In order to manage the rights to reserve a resource, a server and more generally an access rights management service may be implemented in order to authorize or not the reservation or the allocation of a resource. The reservation or allocation of a resource is done from a resource availability status. The virtual occupancy status STI′ of the resource 4, 4′ is then switched from “unallocated” to “reserved” or “allocated”. Thus, a staff member of a first establishment viewing the available resources of a second establishment makes a request to reserve a resource 4, 4′ which may, in a second step, be accepted or not by a staff member of the second establishment. When the request is accepted, the staff member of the second establishment can modify the virtual occupancy status STi′ from the value “available” to “reserved” or “allocated”.

According to one embodiment, when a virtual occupancy status STi′ changes from “available” to “reserved” or “allocated”, the staff member performing the operation can enter a patient identifier ID to ensure that the resource will not be assigned to another patient.

According to one embodiment, an additional virtual occupancy status STi′ may be implemented in the invention aimed at confirming an allocation of a resource 4, 4′ which is in a physical occupancy status in the “available” state. Advantageously, the virtual occupancy status STi′ may comprise two resource allocation values including the value “allocated—confirmed” and the value “allocated unconfirmed”. In this mode of operation, medical staff wishing to allocate a resource from a remote terminal such as the terminal T₁ can switch the value of the virtual occupancy status STi′ by means of an interface.

Confirmation of the allocation “allocated—confirmed” can be automatically generated when several messages indicating an “available” physical occupancy status are returned successively by the connected device 2 to the system 1 following the change of virtual occupancy status to “allocated unconfirmed”.

This function makes it possible to avoid cases where a reservation or allocation conflict occurs when a staff member operates the actuator of the connected device 2 associated with a resource in the interval of an allocation request.

At this stage, two embodiments of the invention may be implemented.

In a first embodiment, the “allocated” virtual occupancy status is updated in the database DB₁ recording the virtual occupancy statuses STi′. The connected device 2 receives no status modification and does not change the state feedback of the physical occupancy status STi which is “available”. Thus, in this embodiment, the physical occupancy status STi corresponds to possibly several values of the virtual occupancy status STi′. In this case, the “available” physical occupancy status corresponds to one of the “available and allocated” or “available and unallocated” virtual occupancy status values. According to another example, a physical occupancy status STi may correspond to three values of virtual occupancy statuses “reservation request”, is “available and allocated” or “available and unallocated”.

In a second embodiment, the “allocated” virtual occupancy status is updated in a file or database BD recording the virtual occupancy statuses STi′. The connected device 2 receives a message Mes2 with the new value of the virtual occupancy status STi′ and changes the state feedback of the value of the physical occupancy status STi, for example on the front face of the connected device 2. This may be a lit or unlit diode, a flashing, a color, a value displayed on a screen of the connected device 2.

Thus, in this embodiment, the physical occupancy status STi corresponds to the value of the virtual occupancy status STi′. In this case, the “available” physical occupancy status corresponds to one of the values of the “available and unallocated” virtual occupancy statuses and the “allocated” virtual occupancy status value may correspond to a physical occupancy status STi comprising a predefined state feedback. For example, the diode of the connected device 2 gives a state feedback of the physical occupancy status STi which is thus either on to indicate that the resource 4, 4′ is “available”, or off, indicating that the resource is “awaiting cleaning”, “neutralized”, “occupied” or “out-of-service”, depending on the indication, for example, of another diode. In this example, a flashing of the diode assigned to the physical occupancy status of the “available” value may indicate that the resource is allocated. Thus, a medical staff member entering a room in which a resource is present can immediately see that the resource in question is allocated by a remote medical staff member who has modified the virtual occupancy status STi′. The medical staff member then knows that he can only assign this resource to the patient to whom it has been assigned. According to another example, a connected device 2 provided with a screen is configured to display the identifier of the patient to which the resource has been assigned. The medical staff member can then immediately check that the patient to whom he wishes to assign a resource is indeed the patient to whom this resource has been assigned.

According to another example, a stopping of the flashing of the diode assigned to the physical occupancy status STi of the reservation or allocation of a resource has been canceled. This case may correspond, for example, to the death of the patient to whom the resource has been assigned, while being transported to the establishment of that resource. The on-site medical staff member can then immediately assign said resource 4, 4′ to a new patient by acting on the actuator in order to select the physical occupancy status of the resource to an “occupied” position.

According to another example, a physical occupancy status STi, such as the value “available”, can correspond to three values of virtual occupancy status STi′, such as “reservation request”, “available and allocated” or “available and unallocated”. The system 1 of the invention therefore allows parallel management, on the one hand, of resources via the connected devices to which they are associated and, on the other hand, of resources via virtual occupancy statuses which can be modified in a remote memory via an electronic terminal such as a computer. The system 1 of the invention allows this management in parallel by collecting centralized data in the memory collecting the virtual occupancy statuses STi′. One advantage of the invention is to avoid conflict cases by maintaining pre-established correspondences between the values of the physical occupancy statuses STi and the values of the virtual occupancy statuses STi′. Another advantage is that, after a predefined period of time, a data message is automatically sent to a resource management system 4, 4′ in order to inform that a given resource 4, 4′ is still in the “allocated” virtual occupancy status. Such a message makes it possible to smooth out resource allocations 4, 4′ and prevents forgetting or mishandling. According to an example, the predefined time can be parameterized.

Status Values

The values of the physical occupancy statuses may notably be predefined and selected from a list.

A first value of the physical occupancy status STi is ‘occupied’. This physical occupancy status STi means that a resource 4, 4′ is used or occupied by a patient. When the rotary control of the connected device 2 is in this position, a message Mes1 has been sent to a data server SERV₁ or SERV₃ to modify the value of the virtual occupancy status STi′ to the value “occupied”. The resource 4, 4′ is then identified as unavailable from any communication interface interrogating the file or database DB₁ in which the values of the virtual occupancy status STi′ are recorded.

A second value of the physical occupancy status STi is “available”. This status means that a resource 4, 4′ is available and ready to welcome a new patient. When the rotary control of the connected device 2 is in this position, a message Mes1 has been sent to a data server SERV₁ or SERV₃ to is modify the value of the virtual occupancy status STi to the value “available”. The resource 4, 4′ is then identified as available from any communication interface interrogating a file or database DB 1 in which the values of the virtual occupancy status STi′ are recorded.

A third value of the physical occupancy status STi is “awaiting cleaning”. This status signifies that a resource is being prepared by medical personnel. In the case of a bed, this may be a cleaning, disinfection, changing sheets or an operation of mechanical or electronic configuration of the bed. This can be a pre-adjustment of a bed's straightening or include a computer connection option. When the rotary control of the connected device 2 is in this position, a message Mes1 has been sent to a data server SERV₁ or SERV₃ to modify the value of the virtual occupancy status STi′ to the value “awaiting cleaning”. The resource 4, 4′ is then identified as unavailable from any communication interface interrogating a file or the database DB 1 in which the values of the virtual occupancy status STi′ are recorded. One advantage is that a data message is sent to an asset management system to inform and schedule a maintenance operation such as cleaning or an operation on the resource. Another advantage is that after an initial predefined time, a data message is sent to a resource management system to inform that a resource is still “awaiting cleaning”. One interest is to coordinate different departments within a hospital such as a medical department and a cleaning department. According to an exemplary embodiment, the first pre-defined time may be parameterized.

A fourth value of the physical occupancy status STi is “neutralized”. This physical occupancy status STi means that the connected device 2 is not assignable. This physical occupancy status is used to check that all the conditions for the allocation of a resource are met. The conditions may be sufficient equipment or sufficient personnel. As soon as the status of a resource is “neutralized”, it is not possible for personnel to assign or allocate or use this resource 4, 4′ without prior authorization. When the rotary control of the connected device 2 is in this position, a message Mes1 has been sent to a data server SERV₁ or SERV₃ to modify the value of the virtual occupancy status STi to the value “neutralized”. The resource 4, 4′ is then identified as unavailable from any communication interface interrogating a file or the database DB₁ in which the values of the virtual occupancy status STi′ are recorded. One advantage is that a data message may be sent to a resource management system 4, 4′ or a material management system associated with the use of a resource 4, 4′ or a personnel management system in order to inform and plan an operation aimed at creating operational conditions necessary for the reallocation of a resource. This could be insufficient caregivers or a disinfectant product not available to perform resource cleaning operations. One interest is to coordinate different departments in a hospital such as a medical department and a technical department, a personnel department, etc. Another advantage is to coordinate resource replenishment if the number of connected devices that are not in the “neutralized” position is below a threshold, this informs that the available resource pool is low. Conversely, when many resources are in the neutralized status, notifications or messages may be automatically sent to a user to show the information that an action must be taken. The message may be sent automatically when a given number of resources in the “neutralized” status is crossed or when too long a time has elapsed during which a resource is in a “neutralized” status.

A fifth value of the physical occupancy status STi is “Out of Service”. In this case, the bed or more generally the resource requires a maintenance operation involving service personnel capable of performing a repair or installation of the connected device that may not be properly attached to the resource. When the rotary control of the connected device 2 is in this position, a message Mes1 has been sent to a data server SERV₁ or SERV₃ to modify the value of the virtual occupancy status STi to the value “Out of Service”. The resource 4, 4′ is then identified as unavailable from any communication interface interrogating a file or the database DB₁ in which the values of the virtual occupancy status STi′ are recorded. One advantage is that a data message may be sent to a resource management system 4, 4′ in order to inform and plan a repair operation on the resource or possibly a management of the resource by personnel to route it to a repair or maintenance center. One interest is to coordinate different departments of a hospital such as a medical service and a technical service such as an after-sales service, or purchasing service to count the number of breakdowns, speed up their handling, ensure follow-up operations, etc. Another advantage is to coordinate resource replenishment when a large number of resources are identified as Out of Service.

The invention allows at least 3 different values of physical occupancy status STi to be taken into account among at least the two values: “occupied” and “available”. In the event that only 3 physical occupancy statuses STi are possible, the third physical occupancy status STi may be, for example, a status bringing together the properties of the “Out of Service” or “Awaiting cleaning” statuses. This status specifies more generally that a resource is temporarily unavailable without however being occupied. One interest is in involving specific personnel to carry out an action whose purpose will result in making the resource available and therefore modifying the physical occupancy status.

According to one embodiment, the values of the virtual occupancy statuses STi′ may be identical to the values of the physical occupancy statuses STi. However, according to one embodiment, the values of the virtual occupancy statuses STi′ may comprise other values such as “allocated” or “unallocated”. One interest is to allow viewing from a resource tracking interface such as that of the terminal T₁ or T₂ in FIG. 8 the different types of resources available. This option makes it possible to not generate conflicting allocation cases and allows the various establishments to interact with each other while respecting a hierarchy of rights of allocation of resources.

Right of Access

According to one embodiment, the connected device 2 comprises a module for controlling an access right. This can be a right of access to the functionalities of the connected device 2 such as access to a button, a position of a rotary control, a change of at least one value of the physical occupancy status STi, a menu of a display screen, an attachment means 22, a reset function or assignment of said connected device 2

The control of access authorization rights notably makes it possible to prevent errors or mishandling of the connected device 2.

The control of access rights may be implemented in different ways. According to a first example, the module is arranged at the level of the connected device 2 by means of a button release lock or a protection for which it is necessary to have a specific key. According to a second example, the module is a magnetic badge reader making it possible to identify the cardholder. According to another example, it may be a module allowing the establishment of a near field data link such as NFC, Bluetooth, RFID, etc. According to another example, the connected device 2 comprises a keypad enabling an access code to be entered. The control module may thus comprise electronic components, including a memory and a calculator to compare a numeric value corresponding to an identifier or a password with a reference value. The reference value may be stored in a local memory of the connected device 2 or a remote memory, for example, located within a rights management server, such as the server SERV₂ of FIG. 8. According to one embodiment, the module for controlling an access right may be a biometric authentication means such as a fingerprint reader.

According to one embodiment, the “neutralized” status may only be actuated by a department head, i.e. a staff member having specific access rights. In this case, access to the position of this physical occupancy status STi by a button or by a rotary control or by a keyboard of the connected device 2 is only possible when the identification of the head of department is carried out using the access control module.

Such personnel may also include medical personnel, hospital personnel and/or cleaning personnel.

Single Authentication Service

According to one embodiment, a rights management server, such as the server SERV₂ implements a single authentication service, better known by the expression “Single Sign On”, through the different components of the system 1, such as the connected devices 2, the servers SERV₁ SERV₂ or SERV₃. One advantage is to make it possible to pass on a change of password to all or some of the components of the system 1.

Mapping

According to one embodiment, a program is implemented in order to display the proportions of resources 4, 4′ and their type within an establishment according to the different values of the different virtual statuses STi. The program may be a WEB platform in SaaS (“Software as a Service”) mode, the data of which is hosted on a data server, for example the server SERV₁ or server SERV₃ of FIG. 8. One interest is, for example, to enable visualization of available beds or available ventilators or to initiate necessary maintenance operations.

According to one embodiment, the system of the invention is generates a numerical mapping of a geographical sector such as a city, region, country or continent and makes it possible to display a plurality of georeferenced establishments in the digital mapping. When an establishment is selected, information about the establishment is displayed, including notably the proportion of resources available at the establishment. The program makes it possible to view and select a resource.

According to one embodiment, a numeric selector such as a check box or a numeric button makes it possible to generate a request automatically sent to the management server of an establishment or directly to an email account attached to an individual of an establishment to request the allocation of a resource for a given patient. This request may be accepted or rejected by the medical personnel of an establishment.

Presence Sensor

According to one embodiment, not represented, the device 2 comprises a presence sensor. The presence sensor may be designed and arranged to detect the presence of a person on the bed. The presence sensor may comprise a weight sensor arranged under the bed. The presence sensor may comprise a motion detector, preferably an infrared motion detector. The presence sensor may also comprise an ultrasonic sensor. The presence sensor may comprise an optical sensor such as a camera, preferably an infrared camera, to detect the presence of a human shape on the bed or the presence of a human shape in the room. In one embodiment, the device comprises a removable shutter between a first occluded position and a clearance position. The shutter, in the occluded position, makes it possible to create an occlusion of the optical sensor. The shutter, in the clearance position, clears the light path between the bed and the optical sensor to allow the optical sensor to detect the presence or absence of a person on the bed. The shutter advantageously makes it possible to preserve the privacy of the person on the bed and to protect the optical sensor from shock or dust. In one embodiment, the device comprises actuation means to move the shutter between the clearance position and the occluded position in a controlled manner. The optical sensor may be arranged in the rack of the connected device or may be integrated into the bed frame and connected to the connected device. The device may be configured to automatically actuate the presence sensor under pre-determined conditions, for example after a pre-determined time during is which the mechanical status has not been modified. The device may comprise rails and/or means forming a runner for moving the shutter between the occluded position and the clearance position.

The presence sensor advantageously makes it possible to raise doubts about the presence of a person in the bed, especially when the physical status has not been modified within a predetermined time. Preferably, the optical sensor is configured to recognize a human shape on a bed.

In one embodiment, the connected device comprises means for controlling the physical actuator and for changing the physical state automatically as a function of the data from the presence sensor. For example, the device may be configured to control the actuator so as to modify the physical status selected by the actuator from “occupied” to “awaiting cleaning” when no presence is detected for a predetermined period of time.

Message Transmission

According to one embodiment, the device is configured to limit the frequency of data message transmission within the data network. One advantage is to limit the flow of data within the network of a structure, especially when the number of devices connected to the network is significant.

According to one embodiment, the connected device is configured to receive a frequency set point from the data network. The device is then configured to limit the frequency of the number of data message transmissions within the data network as a function of the set point received. For example, the device may receive a set point limiting the frequency of the number of transmissions to 1 message per minute or to 1 message per 10 minute period.

The server is preferably configured to send a data message comprising such a set point. In one embodiment, the server is configured to transmit such a message to the data network. In one embodiment, the server is also configured to transmit a data message comprising parameter setting data and the connected device is configured to update its parameters as a function of the parameter setting data received. The parameter setting data may comprise, for example, a WiFi password.

In one embodiment, the transmitted data message comprises data generated by the presence sensor representative of the presence of a person on the bed.

In one embodiment, the server may be configured, after receiving a data message from a connected device, to automatically transmit a confirmation message to said connected device via the data network. Depending on the case, the confirmation message may also comprise a frequency set point or parameter setting data. The connected device may also comprise an alarm, configured to automatically transmit an alert if said device does not receive a confirmation message from the server after a pre-determined time after a data message has been sent within the network. In this respect, the device may comprise a means of sound production or a vibrating means. One advantage is that when a connected device has lost its connection to the server, staff are quickly and automatically alerted.

1. A system for tracking the state of a resource comprising:
at least one connected device comprising:
a physical actuator for the activation or the selection of at least one physical occupancy status with which the physical actuator is associated, the at least one physical occupancy status being selected from a set of physical occupancy statuses comprising at least three states among which a first availability status, a second occupancy status and a third status;
a memory for storing data of said at least one connected device of which at least one identifier of said at least one connected device;
a communication interface to exchange or transmit data stored in the memory of said at least one connected device with at least one remote server;
a visual indicator of a chosen physical occupancy status of said resource;
a means of attachment to the resource so as to make said at least one connected device integral with at least one resource;
at least one remote server accessible from at least one data network, said at least one remote server comprising:
a memory comprising a database and/or data files referencing the different resources and/or the different connected devices and virtual occupancy statuses;
at least one calculator to:
extract at least one virtual occupancy status of a resource from said database and/or said data files and transmit a message within the at least one data network comprising at least one virtual occupancy status of at least one resource, and
record at least one change in the virtual occupancy status of a resource in said database from the reception of data comprising a physical occupancy status of the resource, said data being transmitted by the at least one connected device attached to said resource.

2. The system for tracking the state of a resource according to claim 1, wherein said resource is associated with a given establishment and wherein the at least one data network comprises at least one network equipment arranged within said establishment, said network equipment being a proxy or VPN concentrator to collect all of the messages transmitted by a plurality of connected devices.

3. The system for tracking the state of a resource according to claim 1, wherein the data extracted from the database and/or data files comprising the virtual occupancy statuses are transmitted to:
at least one electronic terminal accessible from the at least one data network and/or;
the connected device, the physical occupancy status of which corresponds to the virtual occupancy status.

4. The system for tracking the state of a resource according to claim 1, wherein the physical actuator is a rotary button comprising a visual indicator indicating the selected physical occupancy status.

5. The system for tracking the state of a resource according to claim 1, wherein the physical actuator is a button making it possible to select a physical occupancy state, said button comprising a diode forming a visual indicator making it possible to indicate a state feedback of a modification of a virtual occupancy status.

6. The system for tracking the state of a resource according to claim 1, wherein the at least one connected device comprises an interface allowing access control of an individual, the access control making it possible to engage a modification of the physical occupancy status according to a predefined value among all values of the physical occupancy statuses.

7. The system for tracking the status of a resource according to claim 1, wherein the at least one connected device has a return screen on which at least one datum is displayed, said datum being transmitted by a remote server.

8. The system for tracking the state of a resource according to claim 1, wherein a number of virtual occupancy statuses TO is strictly greater than the number of physical occupancy statuses.

9. The system for tracking the state of a resource according to claim 1, wherein at least one value of a physical occupancy status corresponds to a first value of a virtual occupancy status or a second value of a virtual occupancy status.

10. The system for tracking the status of a resource according to claim 9, wherein an available value of a physical occupancy status corresponds to one of the two values {“allocated”; “unallocated”} of the virtual occupancy status.

11. The system for tracking the state of a resource according to claim 1, wherein an modification in a remote database or a remote file of the value of a virtual occupancy status causes an automatic generation of a request transmitted through at least one data network, to the at least one connected device, said at least one connected device comprising a calculator to generate an electrical set point to light up a diode for a visual indicator of said physical actuator.

12. The system for tracking the state of a resource according to claim 1, wherein the means of attachment of the device connected to a resource comprises two strips cooperating together, which include a first strip comprising loops and the second strip comprising hooks.

13. The system for tracking the status of a resource according to claim 1, wherein the at least one connected device comprises a geolocation module configured to receive a location message of a location position, said location position being recorded in a memory of said at least one connected device and transmitted to a remote entity.

14. The system for tracking the state of a resource according to claim 1, further comprising:
a first server configured to collect the data transmitted by the at least one connected devices of a first data,
a second server accessible from a second data network connected to the first data network, configured to collect part of the data collected by the first server of the connected devices of the first data network.

15. A method for tracking the state of a resource, comprising:
automatically transmitting a data message from a connected device within a data network following a detection of a change of position of an actuator, said message comprising a physical occupancy status of a resource associated with said connected device;
recording at least one change in a virtual occupancy status of a resource in said database from the reception of data comprising a physical occupancy status of the resource;
extracting at least one virtual occupancy status of a resource from said database and transmission of data to an electronic terminal accessible from the data network, said data comprising at least one virtual occupancy status of at least one resource;
modifying at least one change in the virtual occupancy status of a resource in said database from the reception of a command transmitted from an electronic terminal connected to the data network said command comprising at least one value of a virtual occupancy status comprised in a set of values, said set of values corresponding to a value of a physical occupancy status of the resource.
